(19) United States
(12) Patent Application Publication
(10) Pub. No.: US 2010/0286590 A1
(43) Pub. Date: Nov. 11, 2010

(54) IONTOPHORETIC DEVICE WITH IMPROVED COUNTERELECTRODE

(75) Inventor: Emma Amelia DURAND, Jamestown, RI (US)

Correspondence Address: PILLSBURY WINTHROP SHAW PITTMAN, LLP, P.O. BOX 10500, MCLEAN, VA, 22102, US

(73) Assignee: ISIS BIOPOLYMER LLC, Warwick, RI (US)

(21) Appl. No.: 12/776,346

(22) Filed: May 7, 2010

Related U.S. Application Data
(60) Provisional application No. 61/176,719, filed on May 8, 2009.
(60) Provisional application No. 61/304,013, filed on Feb. 12, 2010.
(60) Provisional application No. 61/302,658, filed on Feb. 9, 2010.

(51) Int. Cl. A61N 1/30

(52) U.S. Cl. 604/20; 604/501

(57) ABSTRACT

The present disclosure relates to an iontophoretic device. In one aspect, the device includes a barrier layer between the drug reservoir and wearer. In another aspect, the device includes a counterelectrode opposite the driving electrode relative to the drug reservoir.

### RELATED APPLICATION

This application claims priority to U.S. Provisional Application Ser. No. 61/176,719 filed May 8, 2009, U.S. Provisional Application Ser. No. 61/304,013, filed Feb. 12, 2010, and U.S. Provisional Application Ser. No. 61/302,658 filed Feb. 9, 2010, the entirety of each of which is incorporated herein by reference.

### FIELD OF THE INVENTION

The present application relates to iontophoretic devices.

### BACKGROUND OF THE INVENTION

Iontophoretic devices are known in the art. They are placed on a patient's skin and use charged electrodes to drive charged drug ions from a drug reservoir and into the patient's skin tissue.

Two major shortcomings of current iontophoretic technology are (1) passive transfer of drug ions from the drug reservoir into the patient's skin tissue when the device is inactive, and (2) irritation to the patient's skin tissue because its impedance is used as an element of the circuit between two oppositely charged electrodes of the device.

The present invention seeks to provide a solution for one or both of these shortcomings.

### SUMMARY OF THE INVENTION

One aspect of the present invention provides an iontophoretic drug delivery device with an enhanced electrode construction. The device comprises a base and a drug reservoir containing a supply of charged drug ions. A driving electrode is positioned above the drug reservoir. A counterelectrode is positioned below the drug reservoir opposite the driving electrode.

A control circuit includes a power source. The control unit is coupled to the driving electrode and the counterelectrode, and is operable in a driving mode for applying a potential to the driving electrode of the same polarity as the charge of the charged drug ions and a potential of opposite polarity to the counterelectrode so as to drive the charged drug ions towards the tissue of the wearer.

Another aspect of the invention provides a method of using such a device. The method comprises operating the device in a driving mode by performing the acts comprising: applying, with the control circuit, a potential to the driving electrode of the same polarity as the charge of the charged drug ions; and applying, with the control circuit, a potential of opposite polarity to the counterelectrode. Thus, the charged drug ions are driven towards the tissue of the wearer.

Yet another aspect of the invention provides an iontophoretic drug delivery device for delivering a drug into the tissue of a wearer. The device of this aspect comprises a base, a drug reservoir comprising a supply of charged drug ions, a driving electrode, and a counterelectrode. A control circuit includes a power source. The control circuit is coupled to the driving electrode and the counterelectrode, and is operable to apply a potential of the same polarity as the charge of the charged drug ions to the driving electrode and a potential of opposite polarity to the counterelectrode. The driving electrode and the counterelectrode are (a) coupled with a resistance therebetween such that current is enabled to flow between the driving electrode and the counterelectrode only within the device, and (b) positioned such that application of the respective potentials thereto in the driving mode of the control circuit drives the charged drug ions towards the tissue of the wearer.

Other objects, features, and advantages of the present application will become apparent from the following detailed description, the accompanying drawings, and the appended claims.

### BRIEF DESCRIPTION OF THE DRAWINGS

FIG. 1 is an exploded cross-sectional view of a device constructed in accordance with the present invention, with top and bottom views also included;

FIG. 2 is a cross-sectional view showing the electrodes and drug reservoir in isolation;

FIG. 3 is an exaggerated cross-section of an alternative embodiment;

FIG. 4 is an example of a control circuit for the embodiment of FIG. 3.

FIG. 5 is another example of a control circuit for the embodiment of FIG. 3; and

FIG. 6 shows an iontophoretic device being used in a central IV line procedure.

### DETAILED DESCRIPTION OF THE ILLUSTRATED EMBODIMENT(S) OF THE INVENTION

The Figures illustrate a non-limiting embodiment of an iontophoretic drug delivery device 10 constructed in accordance with the present invention. The device 10 is constructed to deliver drugs into the tissue of a wearer. The basic principles of iontophoretic devices are well-known, and reference may be made to U.S. Patent Publication No. 2009/0048556 and U.S. Publication No. 2009/0299267 A1 for teachings in this regard, the entirety of which are incorporated herein.

The device 10 comprises a base 12. The base 12 is preferably a flexible structure, such as a foam or plastic, and is designed to conform to the body of the patient and lie adjacent the skin. The base 12 has a drug reservoir opening 14 formed therethrough, which contains a drug reservoir 16. The base 12 may have any construction or configuration, and the illustrated embodiment is not intended to be limiting.

The drug reservoir 16 contains a supply of charged drug ions, which may be elemental ions (i.e., the ionic form of an element), molecular ions (i.e., the ionic form of a molecule), complexed ions (i.e., ions of a weakly bonded group of elements/molecules/ions referred to as a complex). In the illustrated embodiment, the reservoir comprises a gel, such as a hydrogel. The drug may be solvated in a solution in charged ionic form along with the polymer for the gel, and upon curing the polymer cross-links and the charged drug ions are stored in the gel. For example, a salt of the drug may be dissolved in the solution, thus providing drug ions with mobility within the ionically conductive solution/gel. The methods by which such drug reservoirs are formed are known and need not be detailed herein. For example, the drug reservoir 16 may simply be the gel as shown, or it may have a more complex structure, such as a partitioned reservoir with an internal membrane for separating and managing ion mobility. The drug reservoir may have any construction or configuration, and the illustrated embodiment is not intended to be limiting.

The term drug may include any bioactive agent, such as pharmaceuticals, vitamins, treatments, elements, etc., and is not limited to just those drugs subject to regulatory approval. As such, the term drug should be interpreted as meaning any agent having a biological effect on the wearer that is transdermally delivered by the device.

A barrier layer 18 is positioned below the drug reservoir so as to be positioned between the drug reservoir and the tissue of the wearer. The barrier layer has the same configuration as or is larger than the drug reservoir 16 and its opening 14 in terms of area. That is, the barrier layer 18 covers the entire drug reservoir 16, thus maintaining its position between the drug reservoir 16 and the wearer's skin. The barrier layer 18 is configured to prevent essentially prevent passive transport of the charged drug molecules therethrough.

In the illustrated embodiment, the barrier layer 18 is a mesh. The mesh may be coated with an electroconductive material, such as, for example, Ag, AgCl, or carbon. The coating may vary based on the specific drug molecule, delivery rate, and other requirements. The mesh may have any pore size, such as, for example, between 7 and 100 microns. The pore size may also vary depending on the specific drug molecule, delivery rate, and other requirements.

The barrier layer 18 in the illustrated embodiment is formed as part of a layer 20 of the device 10, which is adhered or otherwise bonded to the bottom surface of the base 12. This layer 20 is not necessary, nor is it necessary to form the barrier layer 18 as part of the layer 20.

An adhesive layer 28 may be coated about the peripheral edge of the layer 20. The adhesive layer is preferably a high tack adhesive for firmly bonding the device 10 against the patient's skin. By extending the adhesive to the peripheral edge of the retainer 20 and device 10, the adhesive serves to discourage lifting or peeling of the edges of the device 10, thus maintaining it securely fastened to the skin. Other suitable attachment means may be used to secure the device to a patient, such as tape, straps, etc.

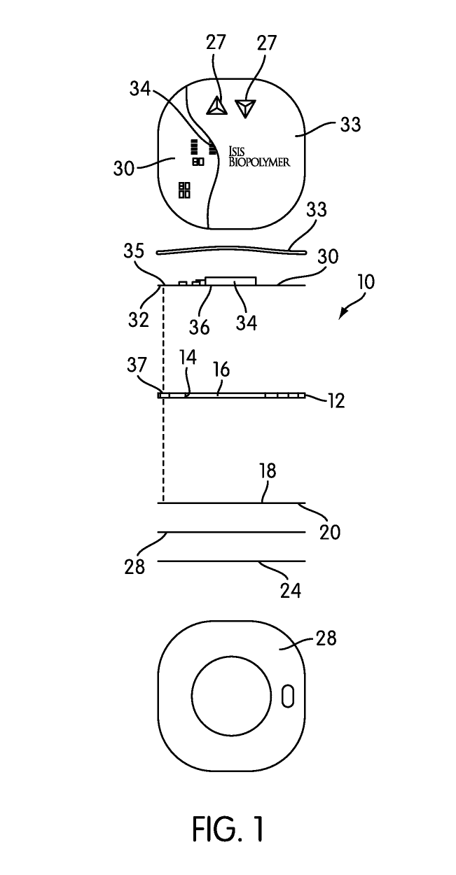

An optional release liner 24 covers the entirety of the bottom surface of the device 10. That is, the release liner 24 covers both the adhesive 28 and may also cover the area of the drug reservoir 16. The release liner 24 may be paper, plastic or another material, and the upper side of the release liner 24 has a release material, such as silicone or wax, so it can be peeled off to expose the adhesive layer 28 and the drug reservoir 16. The release liner 24 is omitted in the bottom view of FIG. 1 so the drug reservoir area can be seen.

Turning to the portions of the device 10 above the base 12 and the drug reservoir 16, the device 10 further comprises a circuit layer 30. The circuit layer is preferably formed of a dielectric (i.e., electrically insulative) substrate 32, such as a flexible non-conductive polymer substrate that can flex to conform to various parts of the patient's body. The upper surface of the substrate 32 includes circuitry, formed preferably as a printed circuitry deposited by polymer thick film coating. That coating technique is disclosed in the above-referenced US Patent Publication 2009/0048556, which may be referred to for its teachings in that regard.

The upper surface of the substrate 32 also includes a power source in the form of a battery. Preferably, the battery may be of the printed type, also taught in US Patent Publication No. 2009/0048556, although any type of battery/power source may be used. A microprocessor 34 is also mounted to the upper surface of the substrate 32, and is coupled to the circuitry and power source for controlling the delivery of electrical power.

Collectively, the circuitry, microprocessor and the power source may be considered a control circuit that controls the application of potentials to the electrodes used in the device 10, which are discussed below. The microprocessor may be omitted, and switches may be used in the control circuit for controlling current flow/direction and the application of the various potentials to the electrodes. Thus, the term control circuit is a structural term that encompasses any circuit coupled to the electrodes for applying potentials thereto, including circuits with or without a microprocessor, integrated circuits, and/or switch-operated circuits.

An optional cover layer 33 is affixed to the substrate 32 to cover and protect the components provided on substrate 32. The cover layer 33 is partially shown in the top view of FIG. 1 to show its relationship to the components, and typically covers all the components.

The device 10 may include one or more activation switches coupled to the control circuit, such as shown at 27. For example, there may be two switches: an on switch for activating the driving mode (as discussed below), and an off switch for stopping the driving mode or activating a forced inactive mode (as discussed below). The switches may be of any type, including membrane switches, button switches, contact switches, piezoelectric, or any other type.

A through-hole (not shown) is formed through the substrate 32 and enables the circuitry on the upper surface of the substrate 32 to be connected to a driving electrode 36 provided on the bottom surface of the substrate 32. The driving electrode 36 is also referred to in the art as a donor electrode. Preferably, the driving electrode 36 is also printed on the bottom surface of the substrate 32 using the polymer thick film coating technique mentioned above. A printed lead may extend from the through-hole to the electrode 36, depending on the placement of the electrode 36 relative to the through-hole. A flexible conductive cement, such as epoxy, may fill the through-hole and connect the circuit on the upper surface to the driving electrode 36 or its lead, and also prevents the infiltration of water up through the through-hole to the circuit. This couples the driving electrode 36 to the control circuitry, thus enabling the power source to apply a potential thereto. Any other suitable way of coupling the driving electrode 36 to the control circuit may also be used.

The driving electrode 36 is positioned above the drug reservoir 16 opposite the barrier layer 18. The driving electrode 36 preferably has the same size and configuration as the drug reservoir 16 and its opening in terms of area, thus enabling the potential applied to the driving electrode 36 to be exposed to the entire drug reservoir 16. During operation in a driving mode, the control circuit applies a potential to the driving electrode 36 of the same polarity as the charge of the charged drug ions so as to drive the charged drug ions towards and into the tissue of the wearer. That is, if the drug is in the form of a positively charged ion, the driving electrode 36 will have a positive charge applied to it. Because charges of the same polarity repel, the positively charged drug ions will be repelled away from the driving electrode 36 and driven towards the tissue of the wearer's skin for permeation into the skin. Conversely, if the drug ion is in the form of a negatively charged ion, the driving electrode 36 will have a negative charge applied to it, thus similarly repelling and driving the drug ions.

The barrier layer 18 is configured to permit the charged drug ions to be actively transported therethrough in the driving mode via the potential applied to the driving electrode 36. That is, the barrier layer 18 may be constructed such that it normally prevents passive transport of the charged drug molecules, but allows the active driven transport by the electromotive force of the driving electrode 36 to occur.

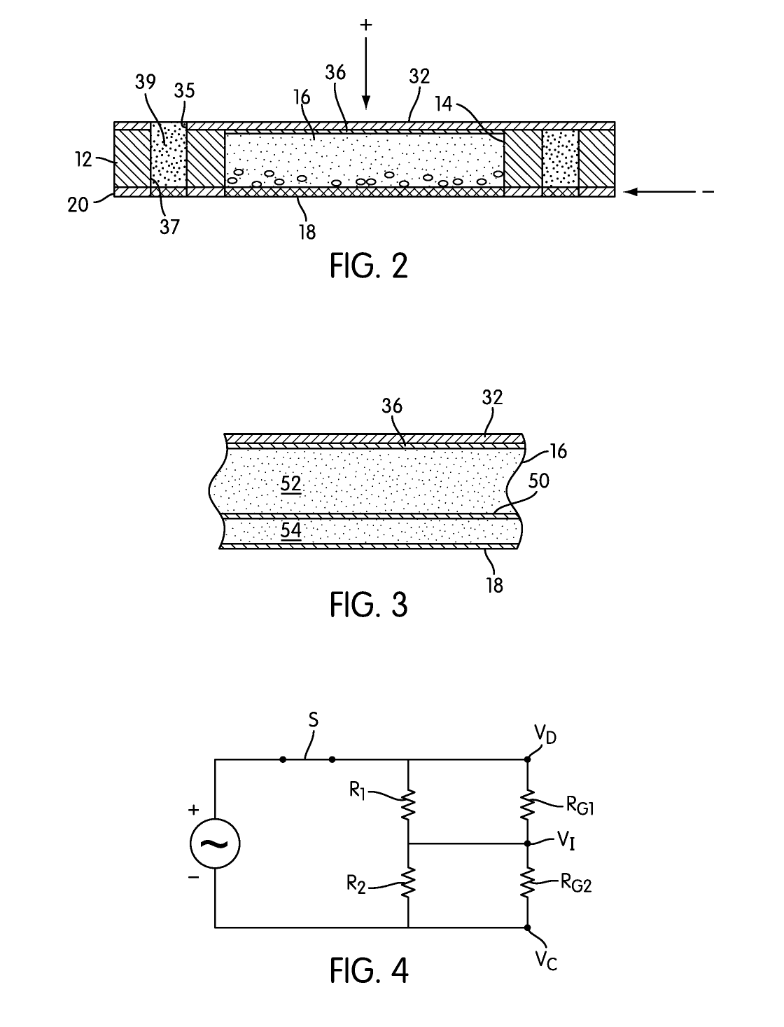

In the illustrated embodiment, the barrier layer 18 is formed of an electroconductive material and is also coupled to the control circuit. To establish the connection, aligned through-holes 35, 37 may be formed through the substrate 32 and the base 12, thus allowing for a lead to couple the barrier layer 18 to the control circuit on the upper surface of substrate 32. A lead may also be formed, such as by polymer thick film printing, on layer 20 depending on the relative placements of the barrier layer 18 and the through-holes 35, 37. For example, the lead may be provided on the upper surface of layer 20 and extend laterally from the through hole 37 to the barrier layer 18, which are spaced laterally apart as shown in FIG. 2. A conductive epoxy 39 may be used as mentioned above to establish the connection between the control circuit and barrier layer 18, and to also prevent water migration. However, any other way of connecting the control circuit to the barrier layer 18 may also be used, including wires, leads, or contacts. The relative sizing of elements in the cross-section of FIG. 2 is somewhat exaggerated to facilitate a better understanding, and different sizes and dimensions may be used.

The control circuit is configured to apply a potential of opposite polarity to the barrier layer 18 so that the barrier layer functions as a counterelectrode. That is, the potential of the driving electrode 18 is applied from one terminal of the power source, whereas the potential of the barrier layer 18 functioning as a counterelectrode is applied from the opposite terminal of the power source. The term counterelectrode specifically refers to and means the electrode that is counter or opposite in charge to the driving electrode 36 and is provided for purposes of completing the iontophoretic circuit between the connections to the opposing terminals of the power source. The microprocessor of the control circuit may be configured to control the application of potentials to both electrodes, and various circuit elements may be used to determine the potential and current density applied to each electrode to ensure proper delivery of the drug molecules.

Preferably, the gel of the drug reservoir 16 is electroconductive, thus completing the circuit comprising the driving electrode 36 and barrier layer/counterelectrode 18. The gel preferably has sufficiently high resistance to maintain a sufficiently high potential difference between the electrodes. Alternatively, rather than rely on the drug reservoir for electroconductively coupling the electrodes, a resistor or other element with a level of resistance may be used to enable the current flow between the electrodes while maintaining an adequate potential difference between the electrodes.

As an example, lidocaine contained in a water-based gel can be delivered using a current density of 0.2 mA/cm² (assuming the driving electrode 36 and counterelectrode/barrier layer 18 have the same area).

Because the barrier layer 18 when used as a counterelectrode will have the opposite polarity as the charged drug molecules in the driving mode, this may enhance the transport of the drug ions. This is because the charged drug molecules will be both repelled away from the driving electrode 36, and attracted towards the barrier layer 18. This may beneficially increase the rate of drug transport achieved per unit power, since both electrodes are contributing to drug transport in the same direction towards the tissue of the wearer.

This construction with the barrier layer 18 serving as the counter-electrode is also advantageous in terms of device size and patient comfort. With prior art devices, typically a counterelectrode is spaced apart laterally from the driving electrode, and the circuit is completed through the impedance or resistance of the patient's skin tissue. While sometimes the counterelectrode can be used with a drug reservoir having drug molecules of a charge opposite the drug in the other reservoir, in many instances only a single drug is being delivered, and thus a “passive” drug-free reservoir is used. In either situation, the device can be irritating because the patient's skin tissue is effectively part of the circuitry, and thus there are practical limits to the power that can be applied to the electrodes. For example, the prior art devices are known to cause burning and “tatooing” (the presence of visible marking) of patent skin. This is a significant drawback of prior art designs. Moreover, if only a single drug is being delivered, a significant portion of the overall area of the device 10 is dedicated to the non-drug delivering passive electrode and reservoir. Even if the counterelectrode is used for purposes of delivering a second drug, it still has the patient discomfort/irritation issue and also is limited to drug ions of the same polarity as its potential (i.e., of charge that is opposite the charge of the drug ions in the other reservoir), thus limiting the potential range of applications that can justify the larger size. With the illustrated embodiment, such an issue is eliminated because there is no laterally spaced counterelectrode that needs to complete the circuit through the wearer's tissue—the circuit is completed within the device with the current flowing between the electrodes across a resistance within the device.

Another advantage is that the gel of the reservoir can maintain a stable conductivity, whereas the conductivity or impedance of skin tissue can vary depending on various conditions, including pH, perspiration, etc. Thus, the device 10 where the barrier layer 18 serves as the counterelectrode eliminates that problem, as the conductivity of the drug reservoir is essentially independent of skin conditions.

Without being limited to a specific mechanism of action, it is believed that the use of the driving electrode and counterelectrode on opposing sides of the drug reservoir creates a high concentration of drug ions at the counterelectrode, which facilitates osmotic transport/permeation of the drug ions into the patient's skin/tissue. Where the counterelectrode is a mesh or permeable membrane, for example, and placed directly against the patient's tissue/skin, this creates intimate contact to further improve such permeation. With prior art devices having the electrodes spaced laterally from one another, the skin itself is part of the “circuit,” as discussed above, and the potential difference is between the electrodes through the skin, which is the primary force in the delivery of the drug. In contrast, the use of the driving electrode and counterelectrode opposing sides of the drug reservoir overcomes the shortcomings of these prior art devices, while still enabling a sufficient amount of drug to be delivered. Indeed, it is possible to use even higher power for the delivery of the drug ion with the opposing driving and counter electrodes because skin is not part of the iontophoretic circuit. This theory of operation is not intended to be limiting. It may be possible in some embodiments that a potential difference could be established between the counterelectrode and the wearer's tissue, which may play a role in drug delivery, but it is believed that any such potential difference would be minor when compared to the controlled larger potential difference between the electrodes within the device itself.

In some embodiments, when it is desired to further minimize the ability of drug ions to passively transport across the barrier layer 18, its polarity may be reversed when the drug is not being delivered. That is, the control circuit may be configured to operate in a “forced inactive” mode and reverse the counterelectrode polarity so that it has the same charge as the drug ions, thus repelling the drug ions away from the patient's skin tissue. Similarly, the polarity of the driving electrode 36 may also be reversed by the control circuit in the forced inactive mode, thus enhancing the repulsive effect of the barrier layer 18 by attracting the drug molecules towards the driving electrode 36 (and hence away from the wearer's skin tissue) by virtue of having the opposite charge as the drug ions. This may be done at a very low power to preserve battery life. This mode of operation may be referred to as forced inactive mode, and the control circuit is configured to be switched to this forced inactive mode to apply these potentials. The term “forced inactive” is used to denote this mode because the device 10 is inactive for delivering the drug, but electrical force is being used to enhance the drug delivery prevention.

In some embodiments, the reversed potentials in this forced inactive mode may be applied to the driving electrode and counterelectrode at predetermined intervals, such as in pulses in accordance with a predetermined duty cycle. This is done to minimize the energy drawn in the inactive mode. Advantageously, the two electrodes when charged will drive the molecules towards to driving electrode 36 and away from the counterelectrode 18 and the wearer's skin tissue. Because passive migration back towards the skin will happen rather slowly, the reversed potentials can be pulsed or intermittently applied to offset that passive migration. Thus, a continuous current draw may not be necessary in the inactive mode. In some embodiments, the counterelectrode may be used irrespective of whether it functions as a barrier layer. That is, the counterelectrode located opposite the driving electrode 36 with the drug reservoir 16 therebetween may be used to minimize or eliminate the flow of current into the user's skin. In such an embodiment, the counterelectrode need not cover the entire bottom surface of the drug reservoir. For example, the counterelectrode may have an annular configuration. Any other construction or configuration may be used.

The device 10 may also have an optional contact sensor to determine that the device is placed properly in contact with a user's tissue. For example, a relatively small contact electrode 42 may be used. This contact electrode 42 may be formed in the same way on layer 20 as the counterelectrode 18. The contact electrode 42 may be coupled to the circuitry on the upper surface of substrate 32 using through-hole connections similarly to the counterelectrode 18. Specifically, aligned through hole 44 and 46 are formed in substrate 32 and base 12, respectively, and filed with an electroconductive material 48, such as an epoxy. The control circuitry on the upper surface of substrate 32 can detect if counterelectrode 18 and contact electrode 42 are in contact with the user's tissue using various techniques. For example, the contact electrode 42 could be set with a polarity opposite the counterelectrode 18 so that establishment of a current flow therebetween can be detected. This may be done by intermittent sampling to prevent continuous current draw, and/or at a very low current flow to prevent tissue irritation.

FIG. 3 is a schematic view of another embodiment of the present invention. Similar components are used, and thus the same reference numbers will be used for components common to this embodiment and the prior one. The device in its entirety is not shown, and only the electrodes and drug reservoir are illustrated, as the device may otherwise be generally the same.

FIG. 3 shows the driving electrode 36, the counterelectrode 18 (which need not be a barrier layer), the drug reservoir 16, and an intermediate electrode 50. The intermediate electrode 50 is positioned between the driving electrode 36 and the counterelectrode 18. Preferably, the intermediate electrode 50 separates the drug reservoir 16 into two portions: a first portion 52 located between the driving electrode 36 and the intermediate electrode 50, and a second portion 54 located between the intermediate electrode 50 and the counterelectrode 18. The intermediate electrode 50 may be disposed in the reservoir 16 in any manner. For example, where the drug reservoir 16 is a gel, the intermediate electrode 50 may be placed in position and set in place as the gel cures. Also, the first and second portions 52, 54 may be separately formed and placed on opposing sides of the intermediate electrode 50. In some embodiments, multiple intermediate electrodes may be used.

The control circuit may be coupled to the intermediate electrode 50 and be operable in the driving mode to apply a potential to the intermediate electrode 50 that is between the potentials applied to the driving electrode 36 and the counterelectrode 18 so as to drive the charged drug ions from the first portion 52 of the drug reservoir 16 into the second portion 54 of the drug reservoir 16 and drive the charged drug ions in the second portion 54 of the drug reservoir 16 towards the tissue of the wearer. That is, the potential difference between the driving electrode 36 and the intermediate electrode 50 is such that, for the drug ions in the drug reservoir's first portion 52, the driving electrode 36 has the same polarity as the charged drug ions and the intermediate electrode 50 has the opposite polarity, thus driving the charged drug ions in the drug reservoir's first portion 52 towards the second portion 54. Similarly, the potential difference between the intermediate electrode 50 and the counterelectrode 18 is such that, for the drug ions in the drug reservoir's second portion 54, the intermediate electrode 50 has the same polarity as the charged drug ions and the counterelectrode 18 has the opposite or counter polarity, thus driving the drug ions from the drug reservoir's second portion 54 towards the wearer's tissue in the same manner as described above. (It should be noted that “polarity” is relative to an opposite or counter electrode, and thus it is correct to state that the intermediate electrode 50 has one polarity (e.g., positive) when compared to the driving electrode 36 and an opposite polarity (e.g., negative) when compared to the counterelectrode 18.)

Without being limited to a specific mechanism of action, it is believed that the drug ions migrate from the first portion 52 of the drug reservoir 16 to the second portion 54 by a “push-pull” action. Specifically, the drug ions in the first portion 52 are driven to the intermediate electrode 50, which is essentially the interface between the first and second reservoir portions 52, 54, by the potential difference between the driving and intermediate electrodes 36, 50. At this interface, the potential difference between the intermediate electrode 50 and the counterlectrode 18 further drives the drug ions away from the intermediate electrode 50 and towards the counterelectrode 18 and patient tissue. Thus, at the interface provided by the intermediate electrode 50, the drug ion migration or transport may be described as being “pushed” towards and then “pulled” away from the intermediate electrode 50 by the potential differences relative to the driving electrode 36 and counterelectrode 18, respectively.

The resistances between the driving electrode 36/intermediate electrode 50 and the intermediate electrode 50/counterelectrode 18 pairs through which current flow is established may be provided by the material of the drug reservoir 16, such as an electroconductive gel, or other resistors, as discussed above. This also enables current flow from the driving electrode 36 to the counterelectrode 18.

Preferably, the spacing between the counterelectrode 18 and the intermediate electrode 50 is less than the spacing between the intermediate electrode 50 and the driving electrode 36. This provides various advantages in both the driving mode, a passive mode, and a forced inactive mode (if used).

The rate at which a drug ion is transported in an ionically conductive drug reservoir is a function of the potential difference between the electrodes on opposing sides of the reservoir, as well as the distance between the electrodes. Thus, from a power efficiency standpoint, closely spaced electrodes are more efficient. However, narrowing the gap between the electrodes also reduces the volume of drug reservoir therebetween (and hence the amount of drug ions stored therein). These are competing factors in the design of a typical iontophoretic device: power efficiency relative to drug delivery rate versus overall volume of drug stored.

With the presence of the intermediate electrode 50, it can be placed closely to the counterelectrode 18 to increase its contribution per unit power to drug delivery rate from the drug reservoir second portion 54, while a larger volume of drug can be stored in the larger first portion 52 between the intermediate electrode 50 and the further spaced driving electrode 36.

Also, the intermediate electrode 50 may be a membrane that reduces or prevents passive transport of the drug ions from the drug reservoir first portion 52 to the second portion 54. This limits the amount of drug ions available for passive absorption into the patient's tissue when the device 10 is not being operated (i.e., the passive mode) to the much smaller amount present in the second portion 54. Even if the intermediate electrode membrane allows some passive transport of the drug ions into the second portion 54, this still acts as an upper limit on the long term passive absorption rate. The counterelectrode 18 may also be constructed as a barrier layer, such as a membrane, as discussed above, to further restrict or prevent passive absorption of the drug ions. Alternatively, the counterelectrode 18 may be open mesh that does not substantially interfere with drug transport.

When either the intermediate electrode 50 or counterelectrode 18 formed as a membrane, it may be formed for any membrane material, including, but not limited to mesh or cloth materials that are metallic or non-metallic, and which may be coated or printed with conductive ink. Preferably the intermediate electrode membrane 50 is hydrophobic to further reduce the transport of drug ions therethrough.

Preferably, the spacing between the intermediate electrode 50 and the counterelectrode 18 is less than or equal to 50% of the spacing between the intermediate electrode 50 and the driving electrode 36. More preferably, that value is less than or equal to 30%, 20%, or 10%. These values are not limiting.

In an embodiment, similarly to the embodiment discussed above, the control circuit is switchable to a forced inactive mode. In this forced inactive mode, the control circuit may at least apply a potential to the counterelectrode 18 of the same polarity as the charge of the charged drug ions and a potential of opposite polarity to the driving electrode 36, thus repelling the drug ions away from the tissue of the wearer. That is, because of the attractive nature of the driving electrode's 36 potential and the repulsive nature of the counterelectrode's 18 potential, the drug ions are encouraged to migrate away from the counterelectrode 18 and the wearer's tissue towards the driving electrode 36.

As an option, the control circuit may also be configured such that in the forced inactive mode the control circuit applies a potential to the intermediate electrode 50 that is between the potentials applied to the driving electrode 36 and the counterelectrode 18. Thus, within the drug reservoir second portion 54, the drug ions are repelled away from the counterelectrode 18 and the wearer's tissue, and attracted towards the intermediate electrode 50; and within the drug reservoir's first portion 52 the drug ions are repelled away from the intermediate electrode 50 and attracted towards the driving electrode 36. Thus, the drug ions are repelled away from the tissue of the wearer and from the drug reservoir second portion 54 to the first portion 52. The same “push-pull” effect may occur at the intermediate electrode 50 as described above, albeit in reverse.

This advantageously uses electrical power to prevent or reduce passive absorption of the drug ions into the wearer's tissue. Preferably, the relatively closer spacing between the counterelectrode 18 and intermediate electrode 50 enhances the rate at which the drug ions are transported within the drug reservoir second portion 54, and the larger spacing between the driving electrode 36 and the intermediate electrode 50 provides increased volume for storage of the drug ions distal from the patient's tissue.

In another embodiment, the control circuit in the inactive mode may apply potentials only to the counterelectrode 18 and the intermediate electrode 50. That is, the control circuit applies a potential to the counterelectrode 18 of the same polarity as the charge of the charged drug ions, and a potential of opposite polarity to the intermediate electrode 50. This repels the drug ions away from the counterelectrode 18 and the tissue of the wearer, and attracts the drug ions to the intermediate electrode 50. Because this will create a high concentration of drug ions at the intermediate electrode 50, some of the drug ions may passively migrate to the first portion 52 of the drug reservoir 16 by osmosis.

In yet another embodiment, the control circuit in the inactive mode may apply potentials only to the intermediate electrode 50 and the driving electrode 36. That is, the control circuit applies a potential to the intermediate electrode 50 of the same polarity as the charge of the drug ions and a potential of opposite polarity to the driving electrode 36. This repels the drug ions away from the intermediate electrode 50, and attracts the drug ions to the driving electrode 36. This prevents or reduces transport of the drug ions into the drug reservoir's second portion 54, thus limiting the amount of drug available for passive absorption into the wearer's tissue. Also, because this will result in low or zero concentration of drug ions in the area of the first drug reservoir portion 52 adjacent the intermediate electrode 50, it is possible (but not necessary) that some drug ions will passively migrate from the second portion 54 to the first portion 52 due to osmosis and the concentration gradient.

In any variation of the forced inactive mode, the respective potentials applied to the electrodes (i.e., either all three electrodes, the driving electrode/counterelectrode pair, the driving electrode/intermediate electrode pair, or the intermediate electrode/counterelectrode pair) may be applied in predetermined intervals, as discussed above.

Although a microprocessor is preferred for precise control of the potentials applied to the electrodes 18, 36, 50, it may be omitted and the control may be provided by basic circuit elements as well.

For example, FIG. 4 shows the basic circuitry for a control circuit with no forced inactive mode. Nodes 18, 36, and 50 represent the counterelectrode, the driving electrode, and intermediate electrode, respectively. Resistors R_{G1} and R_{G2} represent the respective resistances of the gel drug reservoir portions between those electrodes (the G standing for gel, and the 1 and 2 standing for the first and second portions 52, 54 respectively). Resistors R₁ and R₂ constitute a voltage divider for dividing the voltage difference to set the intermediate electrode 50 at an intermediate potential. Switch S, shown in a closed position, connects the power source in the closed position to power the circuit (thus establishing a driving mode) and disconnects the power source in the open position (thus establishing a passive mode).

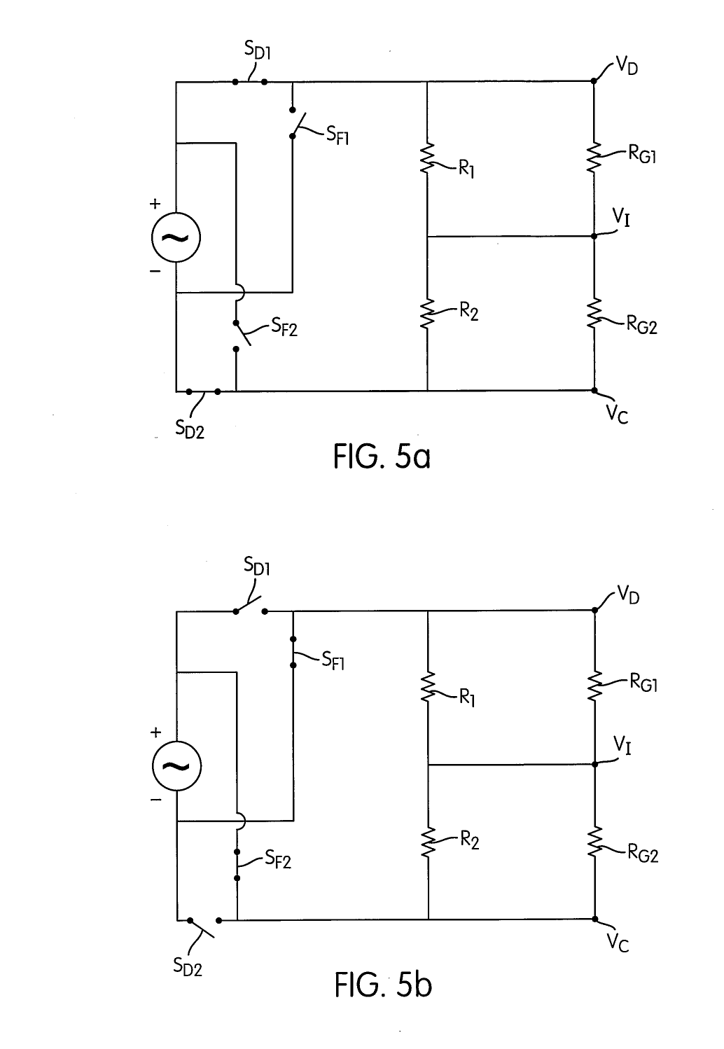

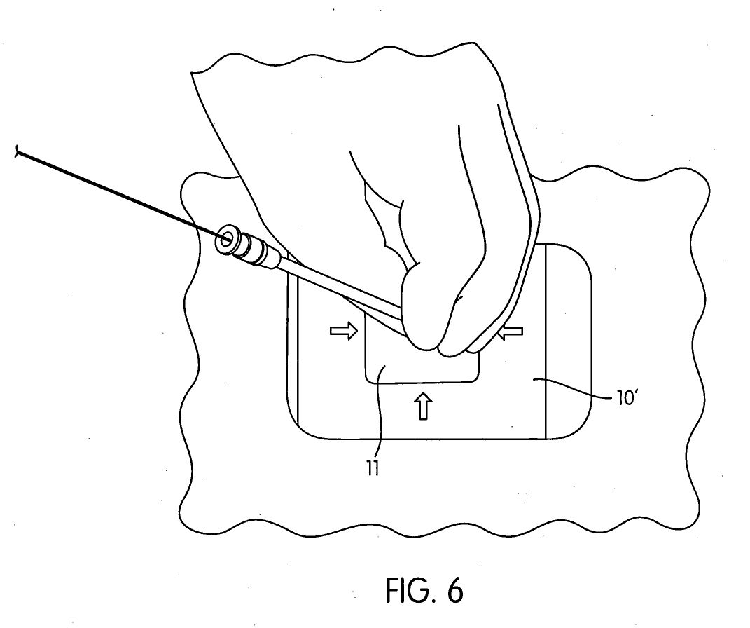

FIGS. 5a and 5b show a circuit similar to FIG. 4, except two pairs of switches S_{D1}/S_{D2} and S_{F1} and S_{F2} are provided. Switches S_{D1}/S_{D2} when closed couple the terminals of the power source in one polarity configuration to establish the driving mode (and the switches S_{F1} and S_{F2} are open), as shown in FIG. 5a. In FIG. 5b, the switch positions are reversed, with switches S_{D1} and S_{D2} open and switches S_{F1} and S_{F2} closed, thus reversing the polarity configuration and establishing the forced inactive mode. In particular, this forced inactive mode has potentials applied to all three electrodes.

In FIGS. 4, 5a, and 5b, V_{D}, V_{I}, and V_{C} schematically denote the nodes at which the driving electrode 36, the intermediate electrode 50, and the counterelectrode 18 are located, and their voltages V_{D}, V_{I}, V_{C} are controlled as described above. Although the example circuits shown are configured for driving positively charged drug ions, the power supply voltage applied can be reversed for driving negatively charged drug ions.

These circuit diagrams are examples only and are not intended to be limiting. Any circuit arrangements may be used.

In one particular application, an iontophoretic device of the present invention may include a procedure window, as shown in U.S. Patent Publication No. 2009/0299267, the entirety of which is incorporated herein. An example of such a device 10′ is shown in FIG. 6, which includes a procedure window 11′. Because the device 10′ can have an opposing electrode construction as described above, it can be made smaller (as it does not need a laterally spaced counterelectrode) or use multiple electrode/reservoir sets for delivering drug ions of the same ionic charge from separate reservoirs (in contrast, prior art laterally spaced electrode designs deliver drugs with ions of opposite ionic charge), or for delivering drugs of different from separate reservoirs. This can be advantageous in a number of different surgical procedures.

One specific surgical procedure is central line insertion. A central line insertion involves the following basic acts:

(1) inserting a hollow needle into a vein (which is typically the femoral, sub-clavian, or jugular vein;

then, if the needle returns suitable blood flow indicating it is properly positioned in the vein:

(2) inserting a guide wire 101 into the vein through the bore in the needle;

(3) retracting the needle along and off the guide wire 101;

(4) disposing a hollow dilator over the guide wire 101;

(5) moving the dilator along the guide wire to penetrate the opening in the patient's skin tissue and vein to dilate the same;

(6) retracting the dilator 102 along and off the guide wire 101;

(7) disposing the catheter 102 over the guide wire 1010;

(8) moving the catheter 102 along the guide wire, through the patient's skin tissue, and into the vein, with a proximal portion of the catheter protruding out from the tissue for access;

(9) retracting the guide wire 101;

(10) dressing the site to secure the catheter against the patient's skin and close the wound (which may include stitching and/or the use of an adhesive dressing).

With the embodiment of FIG. 6, the entire procedure may be performed through the procedure window 11′. It is also possible to place the device 10′ over the catheter 102 and against the patient's tissue around the catheter 102 after the procedure.

The device 10′ may be used to deliver local antibiotics to the procedure site to combat infection. Because central line insertions tend to stay in for a long period of time, infection is a serious issue, and these have a very high infection rate. The key factors driving the high infection rate are (1) the skin is penetrated, and bacteria may penetrate the wound about the catheter, (2) patients requiring central lines typically have one or more serious health conditions, which may weaken their overall immune system response, (3) patients with central lines often are in intensive care units, which tend to be populated with bacteria, particularly bacteria with resistance to general purpose broad spectrum antiobiotics, and (4) many central lines (particularly in emergency room settings) are inserted in the femoral vein at the inner thigh, which tends to have a high prevalence of fecal-related bacteria due to proximity to the rectum. Periodic topical treatments, such as iodine, etc., are used to treat the skin surface, but that generally is less effective against bacteria colonization beneath the skin surface.

With the iontophoretic device of the present invention, the device 10′ can be used to deliver local antiobiotics to the wound site to combat such bacterial colonization and infection. The device 10′ can be left on the patient continuously, and may be programmed to deliver antibiotics on frequent basis. This not only avoids human error or oversight associated with manually applied topical treatments, it also ensures penetration of the drug into the skin tissue itself where topical treatments do not reach. Moreover, the use of local antibiotics targets bacteria at the procedure site where the infection is most likely to occur, unlike oral or intravenous antibiotics which are carried throughout the body by the bloodstream, and may trigger unwanted side effects (such as, for example, disruption of beneficial digestive flora, or vaginal flora that keeps yeast growth in the check).

It is possible for the opposing driving electrode/counterelectrode configuration to be used with the same electrode pair extending about the entire procedural opening 11′. Also, it is possible for discrete sets of opposing driving electrode/counterelectrode pairs to be used. With discrete sets, one or more of the opposing pairs may be used for antibiotic delivery, and one or more of the opposing pairs may be used for delivering a local anesthetic (such a lidocaine). It is also possible for one or more of the pairs to deliver an antibiotic of one type, and another one or more of the pairs to deliver an antibiotic of another type (and more different types could be delivered as well).

In other embodiments, rather than having a procedure window 11′ fully enclosed about its periphery, the window may be open laterally, such as C-shaped, U-shaped, or an opening that is close to be fully enclosed but with a small lateral slot. Such designs may be desired for easy interchanging laterally about the catheter without having to disconnect any delivery device or tubing coupled to the catheter.

The foregoing illustrated embodiments have been provided solely for illustrating the structural and functional principles of the present invention, and should not be regarded as limiting. To the contrary, the present invention is intended to encompass all modifications, substitutions, and alterations within the spirit and scope of the following appended claims.

What is claimed:

1. An iontophoretic drug delivery device for delivering a drug into the tissue of a wearer, the device comprising:
a base;
a drug reservoir containing a supply of charged drug ions;
a driving electrode positioned above the drug reservoir;
a counterelectrode positioned below the drug reservoir opposite the driving electrode;
a control circuit including a power source, the control circuit being coupled to the driving electrode and the counterelectrode and operable in a driving mode for applying a potential to the driving electrode of the same polarity as the charge of the charged drug ions and a potential of opposite polarity to the counterelectrode so as to drive the charged drug ions towards the tissue of the wearer.

2. An iontophoretic device according to claim 1, wherein the counterelectrode is also a barrier layer configured to essentially prevent passive transport of the charged drug ions therethrough.

3. An iontophoretic device according to claim 2, wherein said counterelectrode is a mesh.

4. An iontophoretic device according to claim 1, wherein said control circuit is switchable to a forced inactive mode wherein the control circuit applies a potential to the counterelectrode of the same polarity as the charge of the charged drug ions and a potential of opposite polarity to the driving electrode, thus transporting the drug ions away from the tissue of the wearer.

5. An iontophoretic device according to claim 4, wherein said control circuit is configured such that in said forced inactive mode the respective potentials are applied to the driving electrode and counterelectrode at predetermined intervals.

6. An iontophoretic device according to claim 2, wherein said control circuit is switchable to a forced inactive mode wherein the control circuit applies a potential to the counterelectrode of the same polarity as the charge of the charged drug ions and a potential of opposite polarity to the driving electrode, thus transporting the drug ions away from the tissue of the wearer.

7. An iontophoretic device according to claim 6, wherein said control circuit is configured such that in said forced inactive mode the respective potentials are applied to the driving electrode and counterelectrode at predetermined intervals.

8. An iontophoretic device according to claim 1, wherein said reservoir includes a gel comprising the charged drug ions.

9. An iontophoretic device according to claim 1, wherein said control circuit includes a microprocessor for controlling the application of potentials to the electrodes.

10. An iontophoretic device according to claim 1, wherein said drug reservoir is electroconductive to enable current to flow between the driving electrode and the counterelectrode, thus coupling the driving electrode and counterelectrode and providing a resistance therebetween.

11. An iontophoretic device according to claim 1, wherein said charged drug ions are selected from the group consisting of: elemental ions, molecular ions, and complexed ions.

12. An iontophoretic device according to claim 1, further comprising:
an intermediate electrode positioned between the driving electrode and the counterelectrode within the drug reservoir, a first portion of the drug reservoir being located between the driving electrode and the intermediate electrode and a second portion of the drug reservoir being located between the intermediate electrode and the counterelectrode,
the control circuit being coupled to the intermediate electrode and operable in the driving mode to apply a potential to the intermediate electrode between the potentials applied to the driving electrode and the counterelectrode so as to drive the charged drug ions from the first portion of the drug reservoir into the second portion of the drug reservoir and drive the charged drug ions in the second portion of the drug reservoir towards the tissue of the wearer.

13. An iontophoretic device according to claim 12, wherein said control circuit is switchable to a forced inactive mode wherein the control circuit at least applies a potential to the counterelectrode of the same polarity as the charge of the charged drug ions and a potential of opposite polarity to the driving electrode, thus transporting the drug ions away from the tissue of the wearer.

14. An iontophoretic device according to claim 13, wherein the control circuit is configured such that in said forced inactive mode the control circuit applies a potential to the intermediate electrode between the potentials applied to the driving electrode and the counterelectrode so as to transport the drug ions away from the tissue of the wearer and from the second portion of the drug reservoir to the first portion of the drug reservoir.

15. An iontophoretic device according to claim 12, wherein the control circuit is switchable to a forced inactive mode wherein the control circuit at least applies a potential to the counterelectrode of the same polarity as the charge of the charged drug ions and a potential of opposite polarity to the intermediate electrode, thus transporting the drug ions from the tissue of the wearer.

16. An iontophoretic device according to claim 12, wherein the control circuit is switchable to a forced inactive mode wherein the control circuit at least applies a potential to the intermediate electrode of the same polarity as the charge of the charged drug ions and a potential of opposite polarity to the driving electrode, thus transporting the drug ions in the first portion of the drug reservoir away from the second portion of the drug reservoir and the tissue of the wearer.

17. An iontophoretic device according to claim 12, wherein a spacing between the counterelectrode and the intermediate electrode is less than a spacing between the intermediate electrode and the driving electrode.

18. An iontophoretic device according to claim 13, wherein a spacing between the counterelectrode and the intermediate electrode is less than a spacing between the intermediate electrode and the driving electrode.

19. An iontophoretic device according to claim 14, wherein a spacing between the counterelectrode and the intermediate electrode is less than a spacing between the intermediate electrode and the driving electrode.

20. An iontophoretic device according to claim 15, wherein a spacing between the counterelectrode and the intermediate electrode is less than a spacing between the intermediate electrode and the driving electrode.

21. An iontophoretic device according to claim 16, wherein a spacing between the counterelectrode and the intermediate electrode is less than a spacing between the intermediate electrode and the driving electrode.

22. An iontophoretic device according to claim 13, wherein said control circuit is configured such that in said forced inactive mode the respective potentials are applied to the driving electrode and the counterelectrode of predetermined intervals.

23. An iontophoretic device according to claim 14, wherein said control circuit is configured such that in said forced inactive mode the respective potentials are applied to the driving electrode, the intermediate electrode and the counterelectrode at predetermined intervals.

24. An iontophoretic device according to claim 15, wherein said control circuit is configured such that in said forced inactive mode the respective potentials are applied to the counterelectrode and the intermediate electrode at predetermined intervals.

25. An iontophoretic device according to claim 16, wherein said control circuit is configured such that in said forced inactive mode the respective potentials are applied to the driving electrode and the intermediate electrode at predetermined intervals.

26. An iontophoretic device according to claim 12, wherein said reservoir includes a gel comprising the charged drug ions.

27. An iontophoretic device according to claim 12, wherein said control circuit includes a microprocessor for controlling the application of potentials to the electrodes.

28. An iontophoretic device according to claim 12, wherein said drug reservoir is electroconductive to enable current to flow between the electrodes, thus coupling the electrodes and providing a resistance therebetween.

29. An iontophoretic device according to claim 12, wherein said charged drug ions are selected from the group consisting of: elemental ions, molecular ions, and complexed ions.

30. An iontophoretic device according to claim 1, further comprising:
an intermediate electrode positioned between the driving electrode and the counterelectrode within the drug reservoir, a first portion of the drug reservoir being located between the driving electrode and the intermediate electrode and a second portion of the drug reservoir being located between the intermediate electrode and the counterelectrode,
the control circuit being coupled to the intermediate electrode and being switchable to a forced inactive mode wherein the control circuit applies a potential difference between the intermediate electrode and at least one of the driving electrode and the counterelectrode so as to transport the drug ions away from the tissue of the wearer.

31. An iontophoretic device according to claim 12, wherein the intermediate electrode is also a barrier layer configured to reduce passive transport of the charged drug ions from the first portion of the drug reservoir to the second portion of the drug reservoir.

32. An iontophoretic device according to claim 31, wherein the intermediate electrode has a hydrophobic characteristic.

33. An iontophoretic device according to claim 31, wherein the intermediate electrode is a membrane.

34. An iontophoretic device according to claim 12, wherein the counterelectrode is an open mesh that does not substantially interfere with transport of the drug ions.

35. A method for using an iontophoretic device for delivering a drug into the tissue of a wearer, the device comprising: (i) a base, (ii) a driving electrode positioned above the drug reservoir, (iii) a counterelectrode positioned below the drug reservoir opposite the driving electrode, (iv) a control circuit including a power source, the control circuit being coupled to the driving electrode and the counterelectrode, the method comprising operating the device in a driving mode by performing the acts comprising:
applying, with the control circuit, a potential to the driving electrode of the same polarity as the charge of the charged drug ions; and
applying, with the control circuit, a potential of opposite polarity to the counterelectrode;
wherein the charged drug ions are driven towards the tissue of the wearer.

36. A method according to claim 35, further comprising operating the device in a forced inactive mode by performing the acts comprising:
applying, with the control circuit, a potential to the counterelectrode of the same polarity as the charge of the charged drug ions, and
applying, with the control circuit, a potential of opposite polarity to the driving electrode,
wherein the drug ions are transported away from the tissue of the wearer.

37. A method according to claim 36, wherein said forced inactive mode said potentials are applied to the driving electrode and the counterelectrode at predetermined intervals.

38. A method according to claim 35, wherein the device further comprises an intermediate electrode positioned between the driving electrode and the counterelectrode within the drug reservoir, a first portion of the drug reservoir being located between the driving electrode and the intermediate electrode and a second portion of the drug reservoir being located between the intermediate electrode and the counterelectrode, the control circuit also being coupled to the intermediate electrode, wherein operating the device in the driving mode further comprises:
applying, with the control circuit, a potential to the intermediate electrode between the potentials applied to the driving electrode and the counterelectrode so as to drive the charged drug ions from the first portion of the drug reservoir into the second portion of the drug reservoir and drive the charged drug ions from the second portion of the drug reservoir towards the tissue of the wearer.

39. A method according to claim 38, further comprising operating the device in a forced inactive mode by performing the acts comprising:
applying, with the control circuit, a potential to the counterelectrode of the same polarity as the charge of the charged drug ions,
applying, with the control circuit, a potential of opposite polarity to the driving electrode, and
applying, with the control circuit, a potential to the intermediate electrode between the potentials applied to the driving electrode and the counterelectrode,
wherein the charged drug ions are transported away from the tissue of the wearer and from the second portion of the drug reservoir to the first portion of the drug reservoir.

40. An iontophoretic drug delivery device for delivering a drug into the tissue of a wearer, the device comprising:
a base;
a drug reservoir comprising a supply of charged drug ions;
a driving electrode;
a counterelectrode; and
a control circuit including a power source, the control circuit being coupled to the driving electrode and the counterelectrode, and being operable to apply a potential of the same polarity as the charge of the charged drug ions to the driving electrode and a potential of opposite polarity to the counterelectrode;
the driving electrode and the counterelectrode being (a) coupled with a resistance therebetween such that current is enabled to flow between the driving electrode and the counterelectrode only within the device, and (b) positioned with respect to said drug reservoir such that application of the respective potentials thereto in the driving mode of said control circuit drives the charged drug ions towards the tissue of the wearer.

41. An iontophoretic device according to claim 40, wherein the driving electrode is positioned above the drug reservoir and the counterelectrode is positioned below the drug reservoir opposite the driving electrode.

42. An iontophoretic device according to claim 40, wherein the counterelectrode is also a barrier layer configured to essentially prevent passive transport of the charged drug ions therethrough.

43. An iontophoretic device according to claim 42, wherein said counterelectrode is a mesh.

44. An iontophoretic device according to claim 40, wherein said control circuit is switchable to a forced inactive mode wherein the control circuit applies a potential to the counterelectrode of the same polarity as the charge of the charged drug ions and a potential of opposite polarity to the driving electrode, thus transporting the drug ions away from the tissue of the wearer.

45. An iontophoretic device according to claim 44, wherein said control circuit is configured such that in said forced inactive mode the respective potentials are applied to the driving electrode and counterelectrode at predetermined intervals.

46. An iontophoretic device according to claim 42, wherein said control circuit is switchable to a forced inactive mode wherein the control circuit applies a potential to the counterelectrode of the same polarity as the charge of the charged drug ions and a potential of opposite polarity to the driving electrode, thus transporting the drug ions away from the tissue of the wearer.

47. An iontophoretic device according to claim 46, wherein said control circuit is configured such that in said forced inactive mode the respective potentials are applied to the driving electrode and counterelectrode at predetermined intervals.

48. An iontophoretic device according to claim 40, wherein said reservoir includes a gel comprising the charged drug ions.

49. An iontophoretic device according to claim 40, wherein said control circuit includes a microprocessor for controlling the application of potentials to the electrodes.

50. An iontophoretic device according to claim 40, wherein said charged drug ions are selected from the group consisting of: elemental ions, molecular ions, and complexed ions.

51. An iontophoretic device according to claim 40, further comprising:
an intermediate electrode positioned between the driving electrode and the counterelectrode within the drug reservoir, a first portion of the drug reservoir being located between the driving electrode and the intermediate electrode and a second portion of the drug reservoir being located between the intermediate electrode and the counterelectrode,
the control circuit being coupled to the intermediate electrode and operable in the driving mode to apply a potential to the intermediate electrode between the potentials applied to the driving electrode and the counterelectrode so as to drive the charged drug ions from the first portion of the drug reservoir into the second portion of the drug reservoir and drive the charged drug ions in the second portion of the drug reservoir towards the tissue of the wearer.

52. An iontophoretic device according to claim 51, wherein said control circuit is switchable to a forced inactive mode wherein the control circuit at least applies a potential to the counterelectrode of the same polarity as the charge of the charged drug ions and a potential of opposite polarity to the driving electrode, thus transporting the drug ions away from the tissue of the wearer.

53. An iontophoretic device according to claim 52, wherein the control circuit is configured such that in said forced inactive mode the control circuit applies a potential to the intermediate electrode between the potentials applied to the driving electrode and the counterelectrode so as to transport the drug ions away from the tissue of the wearer and from the second portion of the drug reservoir to the first portion of the drug reservoir.

54. An iontophoretic device according to claim 51, wherein the control circuit is switchable to a forced inactive mode wherein the control circuit at least applies a potential to the counterelectrode of the same polarity as the charge of the charged drug ions and a potential of opposite polarity to the intermediate electrode, thus transporting the drug ions from the tissue of the wearer.

55. An iontophoretic device according to claim 51, wherein the control circuit is switchable to a forced inactive mode wherein the control circuit at least applies a potential to the intermediate electrode of the same polarity as the charge of the charged drug ions and a potential of opposite polarity to the driving electrode, thus transporting the drug ions in the first portion of the drug reservoir away from the second portion of the drug reservoir and the tissue of the wearer.

56. An iontophoretic device according to claim 51, wherein a spacing between the counterelectrode and the intermediate electrode is less than a spacing between the intermediate electrode and the driving electrode.

57. An iontophoretic device according to claim 52, wherein a spacing between the counterelectrode and the intermediate electrode is less than a spacing between the intermediate electrode and the driving electrode.

58. An iontophoretic device according to claim 53, wherein a spacing between the counterelectrode and the intermediate electrode is less than a spacing between the intermediate electrode and the driving electrode.

59. An iontophoretic device according to claim 54, wherein a spacing between the counterelectrode and the intermediate electrode is less than a spacing between the intermediate electrode and the driving electrode.

60. An iontophoretic device according to claim 55, wherein a spacing between the counterelectrode and the intermediate electrode is less than a spacing between the intermediate electrode and the driving electrode.

61. An iontophoretic device according to claim 52, wherein said control circuit is configured such that in said forced inactive mode the respective potentials are applied to the driving electrode and the counterelectrode of predetermined intervals.

62. An iontophoretic device according to claim 53, wherein said control circuit is configured such that in said forced inactive mode the respective potentials are applied to the driving electrode, the intermediate electrode and the counterelectrode at predetermined intervals.

63. An iontophoretic device according to claim 54, wherein said control circuit is configured such that in said forced inactive mode the respective potentials are applied to the counterelectrode and the intermediate electrode at predetermined intervals.

64. An iontophoretic device according to claim 55, wherein said control circuit is configured such that in said forced inactive mode the respective potentials are applied to the driving electrode and the intermediate electrode at predetermined intervals.

65. An iontophoretic device according to claim 51, wherein said reservoir includes a gel comprising the charged drug ions.

66. An iontophoretic device according to claim 51, wherein said control circuit includes a microprocessor for controlling the application of potentials to the electrodes.

67. An iontophoretic device according to claim 51, wherein said charged drug ions are selected from the group consisting of: elemental ions, molecular ions, and complexed ions.

68. An iontophoretic device according to claim 40, further comprising:
an intermediate electrode positioned between the driving electrode and the counterelectrode within the drug reservoir, a first portion of the drug reservoir being located between the driving electrode and the intermediate electrode and a second portion of the drug reservoir being located between the intermediate electrode and the counterelectrode,
the control circuit being coupled to the intermediate electrode and being switchable to a forced inactive mode wherein the control circuit applies a potential difference between the intermediate electrode and at least one of the driving electrode and the counterelectrode so as to transport the drug ions away from the tissue of the wearer.

69. An iontophoretic device according to claim 51, wherein the intermediate electrode is also a barrier layer configured to reduce passive transport of the charged drug ions from the first portion of the drug reservoir to the second portion of the drug reservoir.

70. An iontophoretic device according to claim 69, wherein the intermediate electrode has a hydrophobic characteristic.

71. An iontophoretic device according to claim 69, wherein the intermediate electrode is a membrane.

72. An iontophoretic device according to claim 51, wherein the counterelectrode is an open mesh that does not substantially interfere with transport of the drug ions.
